(19) United States
(12) Patent Application Publication
(10) Pub. No.: US 2019/0209239 A1
(43) Pub. Date: Jul. 11, 2019

(54) A CUTTING ELEMENT FOR A HAIR CUTTING DEVICE

(71) Applicant: KONINKLIJKE PHILIPS N.V., EINDHOVEN (NL)

(72) Inventors: BASTIAAN WILHELMUS MARIA MOESKOPS, UDEN (NL); KIRAN KUMAR THUMMA, EINDHOVEN (NL); RIEKO VERHAGEN, VUGHT (NL); YANNYK PARULIAN JULIAN BOURQUIN, EINDHOVEN (NL); MARK THOMAS JOHNSON, ARENDONK (BE)

(21) Appl. No.: 16/325,551

(22) PCT Filed: Aug. 18, 2017
(86) PCT No.: PCT/EP2017/070907

§ 371 Date: Feb. 14, 2019

(30) Foreign Application Priority Data
Aug. 19, 2016 (EP) 16184989.8
Dec. 6, 2016 (EP) 16202470.7

(51) Int. Cl. A61B 18/20; A61B 18/22

(52) U.S. Cl. CPC A61B 18/203; A61B 2018/00476; A61B 18/22

(57) ABSTRACT

There is provided a cutting element for use in a hair cutting device. The cutting element comprises an optical waveguide having a sidewall, wherein a portion of the sidewall forms a cutting face for contacting hair. The cutting element also comprises a hair support structure configured to restrict lateral movement of a hair in contact with the cutting face, relative to the optical waveguide. The hair support structure comprises a plurality of movement restrictors extending from the cutting face. Each movement restrictor comprises a ridge protruding from the cutting face of the optical waveguide, these ridges are arranged parallel to one another. A hair support structure for use with a cutting element, and a hair cutting device including such a cutting element are also disclosed.

### FIELD OF THE INVENTION

The invention relates to a cutting element for use with a hair cutting device for cutting (e.g. shaving) hair on a body of a subject, and a hair cutting device comprising the same.

### BACKGROUND OF THE INVENTION

A type of shaving device has been proposed in WO 2014/143670 that makes use of laser light. In particular, a laser light source is provided that is configured to generate laser light having a wavelength selected to target a predetermined chromophore to effectively cut a hair shaft. A fibre optic is located on a shaving portion of the device that is positioned to receive the laser light from the laser light source at a proximal end, conduct the laser light from the proximal end toward a distal end, and emit the light out of a cutting region of the fibre optic and toward hair when the cutting region is brought in contact with the hair.

An alternative type of laser shaving device has been proposed in US 2015/359592. The laser shaving device according to this document utilizes a laser beam in open air which is directed substantially parallel to the skin of the user. Hairs passing through the laser beam are cut via a melting process. In order to prevent the laser light to enter into the skin a spacer or distancing element is used to space the skin away from the laser beam travelling in open air.

### SUMMARY OF THE INVENTION

To achieve a clean cut through a hair, the cutting element of the shaving device (i.e. the fibre optic in the case of the device in WO 2014/143670) needs to be brought very close to, or into contact with, a hair to be cut at a particular position for sufficient time. Since both the fibre optic of the cutting element and the hair to be cut are generally cylindrical in shape, there exists a tendency for the hair to slide across the surface of the cutting element, which can cause the cutting location of the hair to move, result in an inadequate cut.

In order to improve the quality of a cut through a hair and, hence, to improve the cutting ability of the shaving device, the position of the cutting element relative to the hair to be cut should ideally not move while hair is being cut.

Therefore, there is a need for an improved cutting element and hair cutting device that have an improved cutting quality.

According to a first aspect, there is provided a cutting element for use in a hair cutting device, the cutting element comprising an optical waveguide having a sidewall, wherein a portion of the sidewall forms a cutting face for contacting hair; and a hair support structure configured to restrict lateral movement of a hair in contact with the cutting face, relative to the optical waveguide. By restricting lateral movement of the hair relative to the cutting element, the hair may be more cleanly cut, as light coupling out from the cutting face of the optical waveguide will couple into the hair at the same position throughout the cutting process. Consequently, the cutting process may be completed more quickly, which may in turn result in an improved hair cutting or shaving experience for the user.

The hair support structure comprises a plurality of movement restrictors extending from the cutting face. Each movement restrictor may be shaped substantially in a shape selected from a group comprising: conical, cubic, cylindrical, teardrop-shaped and bulbous. Movement restrictors of particular shapes may be advantageous for restricting the movement of hair of particular types and sizes. Bulbous movement restrictors may capture or trap hairs to restrict movement of the hairs during cutting.

Each movement restrictor comprises a ridge protruding from the cutting face of the optical waveguide, the ridges being arranged substantially parallel to one another. Each ridge may comprise a structure shaped substantially in a shape selected from group comprising: a cuboid, a triangular prism and a half cylinder.

A separation between adjacent movement restrictors may be between around 20 micrometres and 500 micrometres. In some embodiments, the separation between adjacent movement restrictors may be between around 50 micrometres and 400 micrometres. The separation may be selected based on the thickness (i.e. diameter) and/or type of hair to be cut.

In use, the hair support structure may be arranged to engage skin of the user from which extends the hair to be cut. Thus, in addition to restricting movement of hairs relative to the optical waveguide, the movement restrictors serve to manipulate skin from which the hairs are growing. In particular, the movement restrictors may push down on the skin surrounding the hairs, thereby revealing more of each strand of hair so that a closer shave can be achieved.

In some embodiments, the hair support structure may be coupled to the optical waveguide using adhesive. In other embodiments, the hair support structure may be configured to be connected to the optical waveguide by an attachment assembly. The hair support structure may be removably connectable to the cutting element, such that it can be replaced, for example when a different support structure is needed, or when the hair support structure is damaged and needs to be disposed of.

The optical waveguide may, in some embodiments, comprise an optical fibre.

According to a second aspect, there is provided a hair support structure for use with a cutting element, the cutting element comprising an optical waveguide having a sidewall, wherein a portion of the sidewall forms a cutting face for contacting hair, the hair support structure comprising a plurality of movement restrictors which movement restrictors comprise a ridge protruding from the cutting face of the optical waveguide. These ridges are arranged substantially parallel to one another such that the hair support structure is configured to restrict lateral movement of a hair in contact with the cutting face, relative to the optical waveguide.

In some embodiments, the hair support structure may comprise an attachment assembly for removably attaching the hair support structure to a cutting element or to a hair cutting device including a cutting element. The attachment assembly may comprise a clip for clipping the hair support structure onto a cutting element or a hair cutting device. Thus, the hair support structure may be removable from the cutting element, and replaced with a different hair support structure. In some embodiments, the cutting element and the hair support structure may be formed as a single unit which may be removable from a hair cutting device.

According to a third aspect, there is provided a hair cutting device for cutting hair on a body of a subject, the hair cutting device comprising a light source for generating laser light at one or more specific wavelengths corresponding to wavelengths absorbed by one or more chromophores in hair; and a cutting element coupled to the light source to receive laser light. The cutting element may comprise a cutting element as described above.

In some embodiments, the hair support structure may be removably attachable to the cutting element.

Other advantageous features will become apparent from the following description.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 is a block diagram of a hair cutting device according to an embodiment of the invention;

FIG. 2 is a pair of schematic drawings showing different views of an exemplary hair cutting device according to an embodiment of the invention;

FIG. 3 is a graph illustrating the refractive index of hair;

FIG. 4 is an illustration of an optical waveguide cutting element having an example hair support structure according to embodiments of the invention;

FIG. 5 is an illustration of an optical waveguide cutting element having an example hair support structure according to embodiments of the invention;

FIG. 6 is an illustration of an optical waveguide cutting element having an example hair support structure according to embodiments of the invention;

FIG. 7 is an illustration of an optical waveguide cutting element having an example hair support structure according to embodiments of the invention;

FIG. 8 is an illustration of an optical waveguide cutting element having an example hair support structure according to embodiments of the invention;

FIG. 9 is an illustration of an optical waveguide cutting element having an example hair support structure according to embodiments of the invention; and

FIG. 10 is an illustration of an example hair support structure having an attachment assembly, according to embodiments of the invention.

### DETAILED DESCRIPTION OF THE EMBODIMENTS

As noted above, the present invention provides an improvement in the cutting ability and quality of a laser light-based shaving device, for example as described in WO 2014/143670. In particular, it has been recognised that by restricting the relative movement of a hair and the cutting element during cutting, the hair can be cut more cleanly and effectively, reducing the need for a user of the shaving device to repeatedly use the shaving device over the same area of his or her skin and, consequently, reducing the risk of pain or irritation of the skin.

It will be appreciated that the invention is applicable to shaving devices (e.g. razors or electric shavers), and any other type of device that is used to cut hair (e.g. hair clippers), even if those devices do not necessary aim to provide a ‘clean shave’ (i.e. to remove hair at the level of the skin).

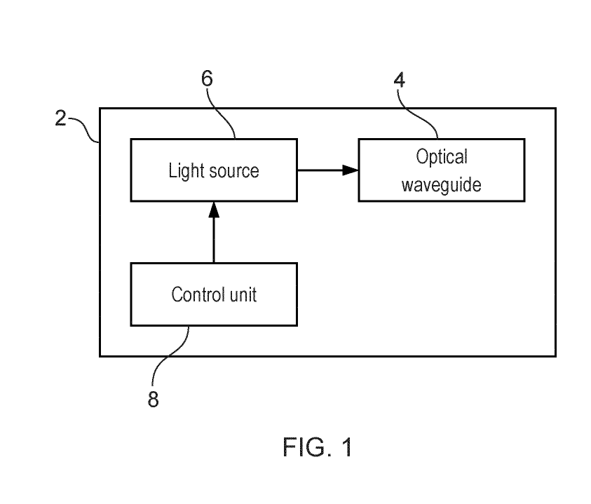

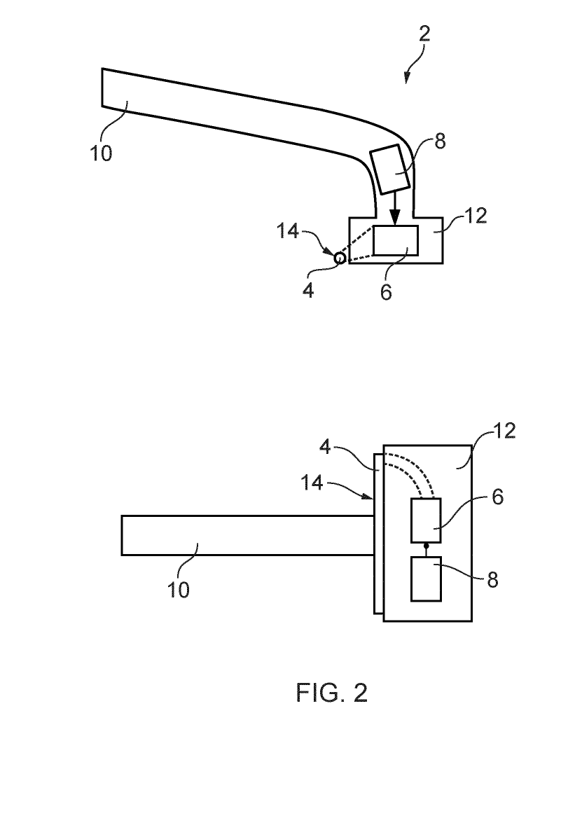

FIG. 1 is a block diagram of a hair cutting device 2 according to an embodiment of the invention. FIG. 2 shows a hair cutting device 2 in the form of a handheld razor according to an exemplary embodiment of the invention. The hair cutting device 2 is for cutting (e.g. shaving) hair on a body of a subject. The subject may be a person or an animal. The hair may be facial hair (i.e. hair on the subject's face), or hair on the subject's head or other part of their body (legs, chest, etc.).

The hair cutting device 2 comprises a cutting element 4 that enables hair to be cut as the hair cutting device 2 is moved over the skin of a subject. The cutting element 4 is an optical waveguide 4 that is arranged on the hair cutting device 2 so that the optical axis of the optical waveguide 4 (i.e. the line along which light typically propagates through the optical waveguide 4) is generally perpendicular to the direction in which the hair cutting device 2 is moved so that hairs contact the sidewall of the optical waveguide 4 (the sidewall corresponding to the long edge of the optical waveguide 4) as the hair cutting device 2 is moved across the skin of the subject. In some embodiments, the optical waveguide 4 is an optical fibre, although those skilled in the art will be aware of other types of optical waveguide that can be used according to the invention, such as a slab waveguide, a strip waveguide or a photonic crystal waveguide. An optical fibre comprises a core, and in some embodiments also comprises a cladding, which may or may not fully encompass the core (e.g. part of the core may be exposed).

A light source 6 is provided in the hair cutting device 2 that generates laser light at one or more specific wavelengths. The light source 6 is optically coupled to the optical waveguide 4 so that the laser light generated by the light source 6 is coupled into the optical waveguide 4 (and specifically coupled into an end of the optical waveguide 4 so that the laser light propagates through the optical waveguide 4).

The light source 6 is configured to generate laser light at one or more specific wavelengths that can be used to cut or burn through hair. In particular, each wavelength corresponds to the wavelength of light absorbed by a chromophore that is found in hair. As is known, a chromophore is the part of a molecule that provides the molecule with its colour. Thus, the laser light will be absorbed by the chromophore and converted into heat which will melt or burn the hair or otherwise destroy the bonds in the molecules of the hair, and it is this melting or burning that provides the cutting action of the hair cutting device 2.

Suitable chromophores that can be targeted by the laser light generated by the light source 6 include, but are not limited to, melanin, keratin and water. Suitable wavelengths of laser light that can be used include, but are not limited to, wavelengths selected from the range 380 nm (nanometres) to 500 nm and 2500 nm to 3500 nm. Those skilled in the art will be aware of the wavelengths of light that are absorbed by these chromophores, and thus also the specific wavelengths of light that the light source 6 should generate for this purpose, and further details are not provided herein.

In some embodiments the light source 6 can be configured to generate laser light at a plurality of wavelengths (either simultaneously or sequentially), with each wavelength being selected to target a different type of chromophore. This can improve the cutting action of the optical waveguide 4 since multiple types of molecules in the hair may be burnt using the laser light. Alternatively, multiple light sources 6 can be provided that each generate laser light at a respective wavelength, and each light source 6 can be coupled to a respective optical waveguide 4 to provide multiple cutting elements 4 in the device 2.

The hair cutting device 2 also comprises a control unit 8 that controls the operation of the hair cutting device 2, and in particular is connected to the light source 6 to control the activation and deactivation of the light source 6 (and in some embodiments control the wavelength and/or intensity of the light generated by the light source 6). The control unit 8 may activate and deactivate the light source 6 in response to an input from a user of the hair cutting device 2. The control unit 8 can comprise one or more processors, processing units, multi-core processors or modules that are configured or programmed to control the hair cutting device 2.

As noted above, FIG. 2 shows a hair cutting device 2 that is in the form of a handheld wet razor. FIG. 2 shows a side view and a bottom view of the razor 2. The razor 2 comprises a handle 10 for the subject (or other user of the device 2) to hold, and a head portion 12 that includes the cutting element 4 (optical waveguide/fibre). As shown, the optical waveguide 4 is arranged along an edge of the head portion, and a part of the optical waveguide 4 forms (or corresponds to) a cutting face 14. The cutting face 14 is the part of the optical waveguide 4 that is intended to come into contact with hair as the hair cutting device 2 is moved across the skin of the subject. A light source 6 and control unit 8 are shown as being incorporated into the head portion 12 and handle 10 respectively, but it will be appreciated that the positions of these components in the hair cutting device 2 as shown in FIG. 2 is not limiting. Likewise it will be appreciated that the embodiment shown in FIG. 2 is merely an example, and the invention can be incorporated or used in any type of hair cutting device 2 that comprises an optical waveguide cutting element 4 as described herein.

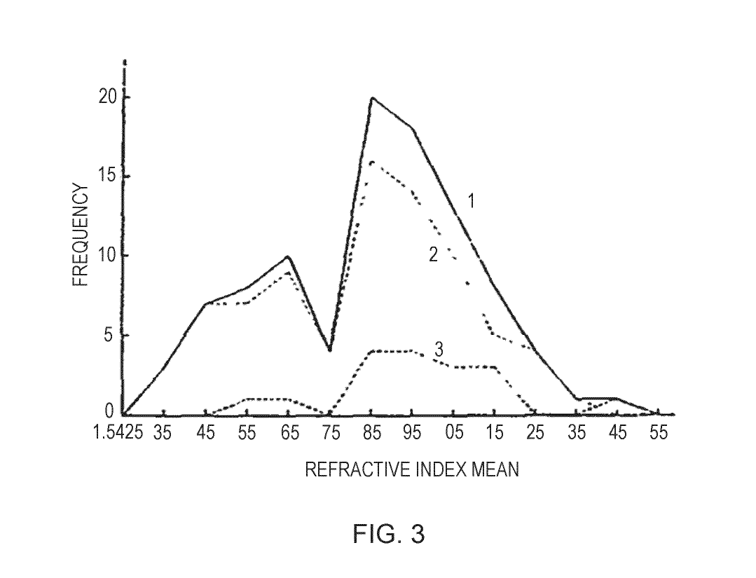

The graph in FIG. 3 illustrates the refractive index of hair, which can be found in a paper by M. D. Greenwell, A. Willner, Paul L. Kirk: Human Hair Studies: III. Refractive Index of Crown Hair, 31 Am. Inst. Crim. L. & Criminology 746 (1940-1941). Curve 1 is a composite line, curve 2 is a line representing the refractive index for Caucasian people, and curve 3 is a line representing the refractive index for non-Caucasian people. Thus, it can be seen that the refractive index of hair is between (approximately) 1.545 and 1.555, although there will be variation between individuals. For example the above paper also recognises that the refractive index of hair can depend on the sex of the subject, e.g. the refractive index of hair on a female is generally higher than the refractive index of hair on a male.

As is known, the optical waveguide 4 acts as a waveguide for the light coupled from the light source 6 through the occurrence of total internal reflection, since the refractive index of air is lower than that of the optical waveguide 4. However, if an object that has a refractive index higher than the optical waveguide 4 is put into contact with the optical waveguide 4, then the total internal reflection is ‘frustrated’ and light can couple from the optical waveguide 4 into that object. Thus, in order for light to be coupled into a hair from the optical waveguide 4 (to provide the cutting action according to the invention), the optical waveguide 4 should preferably have the same or a lower refractive index than hair at the point at which the hair contacts the optical waveguide 4. Thus, the optical waveguide 4 should preferably have the same or a lower refractive index than hair at least at the cutting face 14 portion of the optical waveguide 4. Preferably the refractive index of the optical waveguide 4 at the cutting face 14 is the same as that of hair since that provides the best coupling of light from the optical waveguide 4 to the hair. Light may still be able to couple from the optical waveguide 4 into an object (e.g. a hair) brought into contact with the cutting face 14 of the optical waveguide even if the refractive index of the optical waveguide is higher than that of the object, due to a high numerical aperture in the cutting face.

Thus, in some embodiments, the refractive index of the optical waveguide 4 at least at the cutting face 14 is equal to or lower than 1.56. More preferably the refractive index of the optical waveguide 4 at least at the cutting face 14 is equal to or lower than 1.55. Even more preferably, the refractive index of the optical waveguide 14 at least at the cutting face 14 is equal to or lower than 1.54, since this refractive index is below the refractive indices identified in FIG. 3.

In some embodiments, a lower bound for the refractive index of the optical waveguide 4 at the cutting face 14 can be 1.48, 1.51, 1.53 or 1.54.

A range of values from which the refractive index of the optical waveguide 4 is selected can be formed from any combination of the upper and lower refractive index bounds set out in the preceding paragraphs.

The optical waveguide/fibre 4 can be made from any suitable material or combination of materials. For example optical waveguides/fibres can be composed of or comprise silica, fluoride glass, phosphate glass, chalcogenide glass, crown glass (such as BK7) and/or crystals (such as yttrium aluminium garnet (YAG) or sapphire).

As noted above, the optical waveguide/fibre 4 and strands of hair to be cut by the cutting device are generally cylindrical in shape, and the optical waveguide and a strand of hair are typically oriented such that they are approximately perpendicular to one another when cutting is taking place. Therefore, when the optical waveguide 4 and the strand of hair come into contact with one another, there is a chance that the hair will move along the surface of the optical waveguide such that the position at which the hair is being cut by the optical waveguide changes during the cutting procedure. Therefore, it has been recognised that supporting hair to restrict its movement relative to the cutting element during cutting helps to achieve a cleaner and more effective cut.

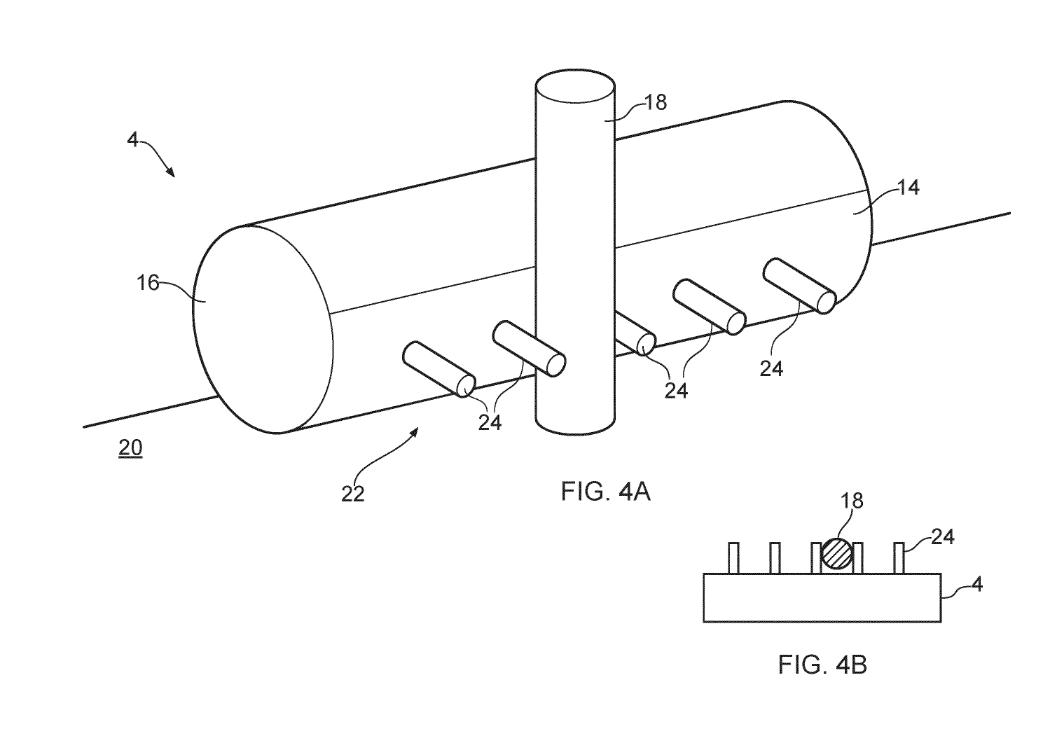

FIGS. 4 to 10 illustrate exemplary embodiments of the cutting element 4 (optical waveguide 4) according to the invention. In FIGS. 4A, 5A, 6A, 7A, 8A, 9A and 10, only a portion of the optical waveguide 4 part of the hair cutting device 2 is shown, and in each of those Figures the optical waveguide 4 is shown in perspective view. No support structure for the optical waveguide 4 is shown. FIGS. 4B, 5B, 6B, 7B, 8B and 9B show the optical waveguides of FIGS. 4A, 5A, 6A, 7A, 8A and 9A, respectively, in plan view.

In FIGS. 4 to 10, an optical waveguide 4 is shown that has a core 16. In the illustrated embodiments, the optical waveguide 4 does not include any cladding around the core 16. However, it will be appreciated that in some embodiments the optical waveguide 4 can comprise cladding around the core 16, although preferably no cladding is present along the cutting face 14 (and indeed, in some embodiments the cutting face 14 can correspond to those parts of the optical waveguide 4 where there is no cladding).

In each of the embodiments shown in FIGS. 4 to 10, the optical waveguide 4 is shown in contact with a hair 18 and the skin 20. The portion of the sidewall of the core 16/optical waveguide 4 that is intended to contact hairs during use forms the cutting face 14. As described above, the refractive index of the core 16 is the same or lower than the refractive index of hair.

The core 16 may have a uniform refractive index (i.e. the same refractive index throughout the core 16), or it may be a graded index fibre, which means that the refractive index decreases with increasing distance from the optical axis.

In each of FIGS. 4 to 10, the cutting element 4 further includes a hair support structure 22 configured to support strands of hair 18 while they are being cut by the cutting element 4. FIGS. 4 to 10 show various forms that the hair support structure 22 may take according to different embodiments.

FIG. 4 shows a cutting element 4 having a hair support structure 22 which comprises a plurality of movement restrictors extending from the cutting face 14. In this embodiment, the movement restrictors are in the form of cylindrical protrusions 24, which extend lengthways from the cutting face 14. The cylindrical restrictors 24 are sized such that, as the cutting element 4 is moved over a surface to be treated, for example the skin of a user, then any hairs protruding from the skin in the path of the cutting element are urged into the space between adjacent protrusions 24. Lateral movement of the hair 18 with respect to the cutting element 4 is restricted, and may be substantially prevented, by the protrusions either side of the hair. In some embodiments, the cylindrical restrictors 24 may have rounded ends or edges to assist with guiding hairs towards the cutting face 14 in the region between adjacent protrusions.

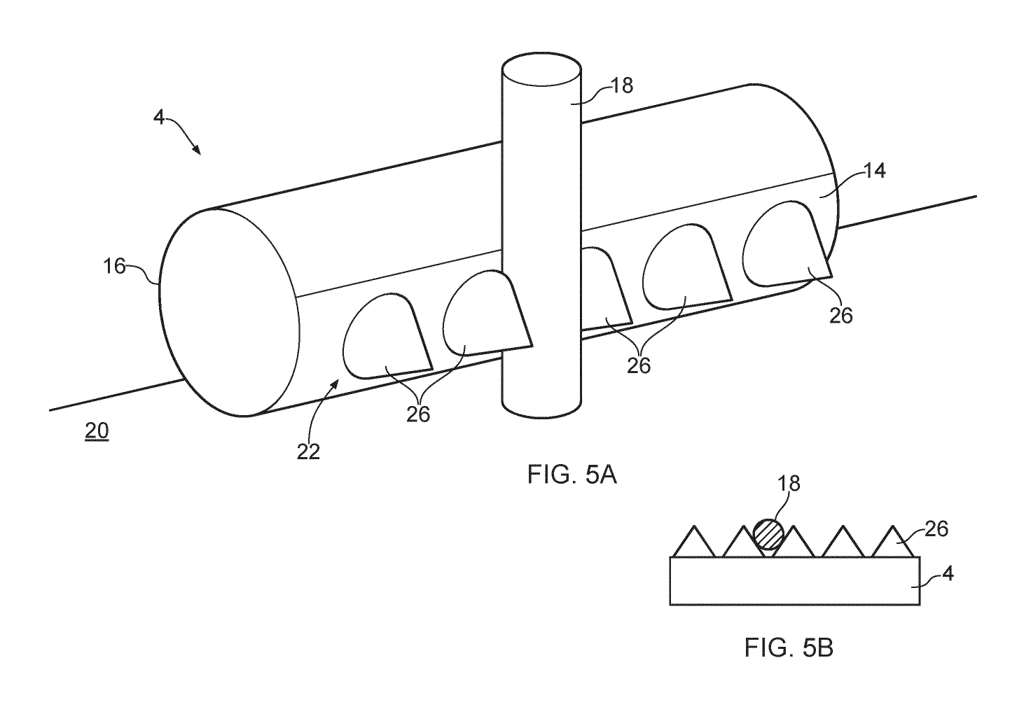

The cutting element 4 shown in the embodiment of FIG. 5 also includes a hair support structure 22 which comprises a plurality of movement restrictors extending from the cutting face 14. The movement restrictors of this embodiment, however, are in the form of conical protrusions 26. In this embodiment, a hair 18 may be guided towards the cutting face 14 in the region between adjacent protrusions 26, and as the hair 18 approaches and/or contacts the cutting face 14, lateral movement of the hair with respect to the cutting element 4 may be restricted. In some embodiments, the movement restrictors may be pyramidal in shape. Edges and/or points of the movement restrictors may be rounded or chamfered to reduce discomfort of a user of a cutting device having such a cutting element 4, in the event the movement restrictors are pushed into the user's skin.

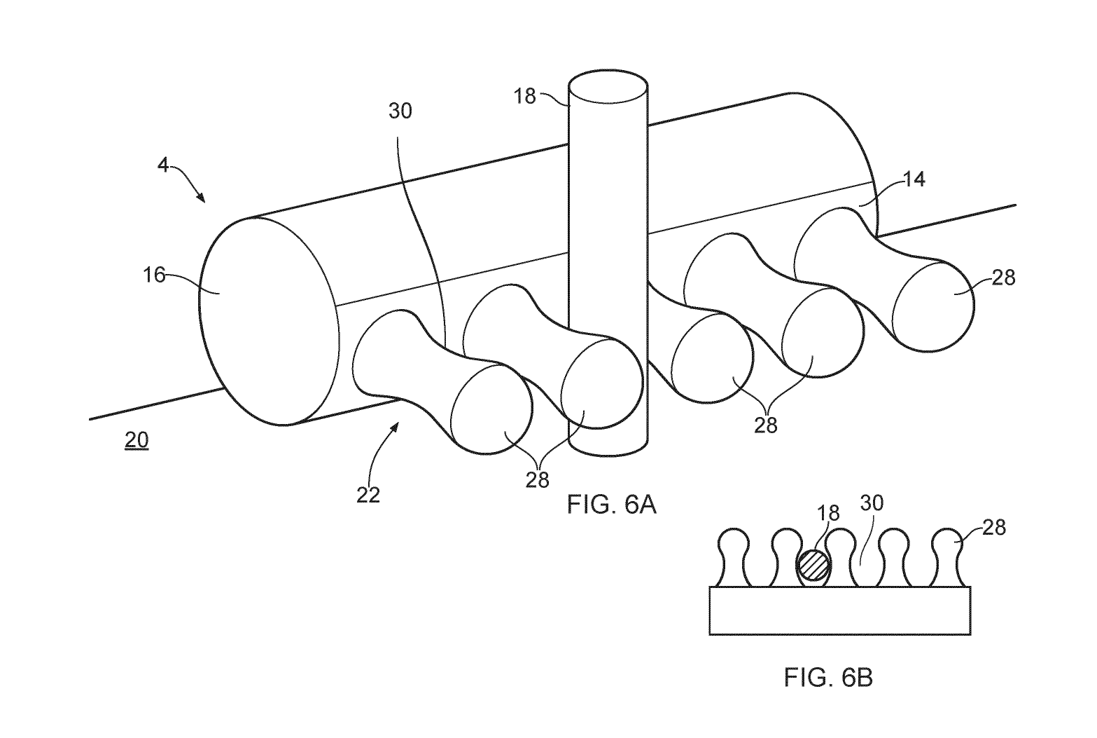

FIG. 6 shows a further example of a cutting element which includes a hair support structure 22 having a plurality of movement restrictors. In this embodiment, the movement restrictors comprise bulbous protrusions 28. A region 30 between adjacent bulbous protrusions 28 serves as a hair-receiving region, and is substantially round in shape. The bulbous protrusions 28 may be formed from resilient material, such that as the hair support structure is moved towards and engages a hair 18, the hair may urge adjacent bulbous protrusions away from one another until the hair enters the hair-receiving region 30. The adjacent bulbous protrusions 28 may then return to their previous position, thereby trapping the hair in the hair-receiving region 30 while it is cut. Since the bulbous protrusions 28 have a rounded shape, they are unlikely to cause irritation when engaging and moving over a user's skin.

It will be appreciated that the movement restrictors may have shapes other that those discussed above. The movement restrictors may be positioned on the cutting face 14 of the cutting element 4 in a spaced apart configuration, with the spacing between adjacent restrictors chosen, in some examples, based on the size (e.g. diameter) of the hair to be cut. For example, in some embodiments, the movement restrictors may be separated by a distance slightly exceeding the diameter of an average hair. In this way, a hair is likely to fit into the space between adjacent restrictors, but further lateral movement with respect to the cutting element 4 is restricted, or prevent altogether.

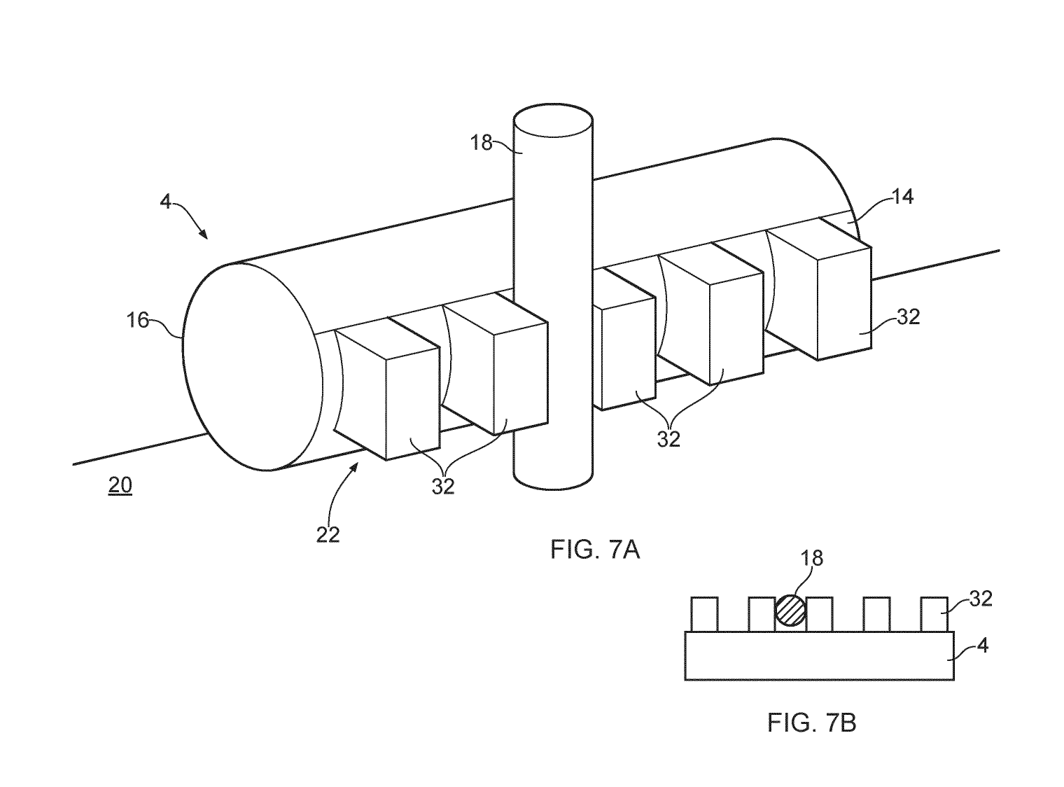

In some embodiments, the hair support structure 22 may comprise structures which function in a similar manner to the movement restrictors discussed above, but which are formed as ridges extending from the cutting face. FIGS. 7, 8 and 9 show embodiments in which the hair support structure 22 comprises ridges of different shapes.

FIG. 7 shows a cutting element 4 having a hair support structure 22 which comprises a plurality of ridges, or ribs, extending from the cutting face 14. In this embodiment, the ridges are in the form of cuboids 32, which extend from the cutting face 14. In this embodiment, the ridges 32 have a square cross section along their longitudinal axis. In other embodiments, however, the ridges may have cross sections of other shapes. In some embodiments, edges of the ridges may be rounded or chamfered to reduce discomfort in the event that the hair support structure was to come into contact with the skin of a user.

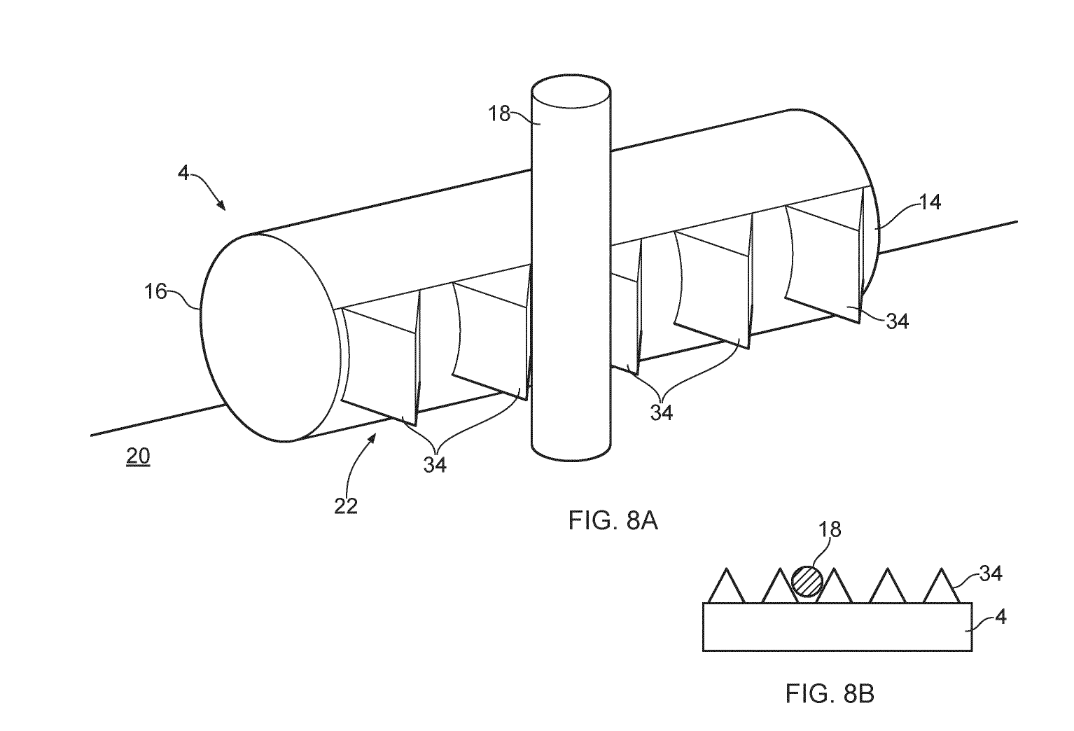

In the embodiment of FIG. 8, the cutting element 4 includes a hair support structure 22 which comprises a plurality of ridges in the form of triangular prisms 34. The triangular prism structures 34 extend from the cutting face 14, and serve to guide a hair 18 towards the cutting face in the region between adjacent ridges. Edges of the prisms 34 may, in some embodiments, be rounded or chamfered.

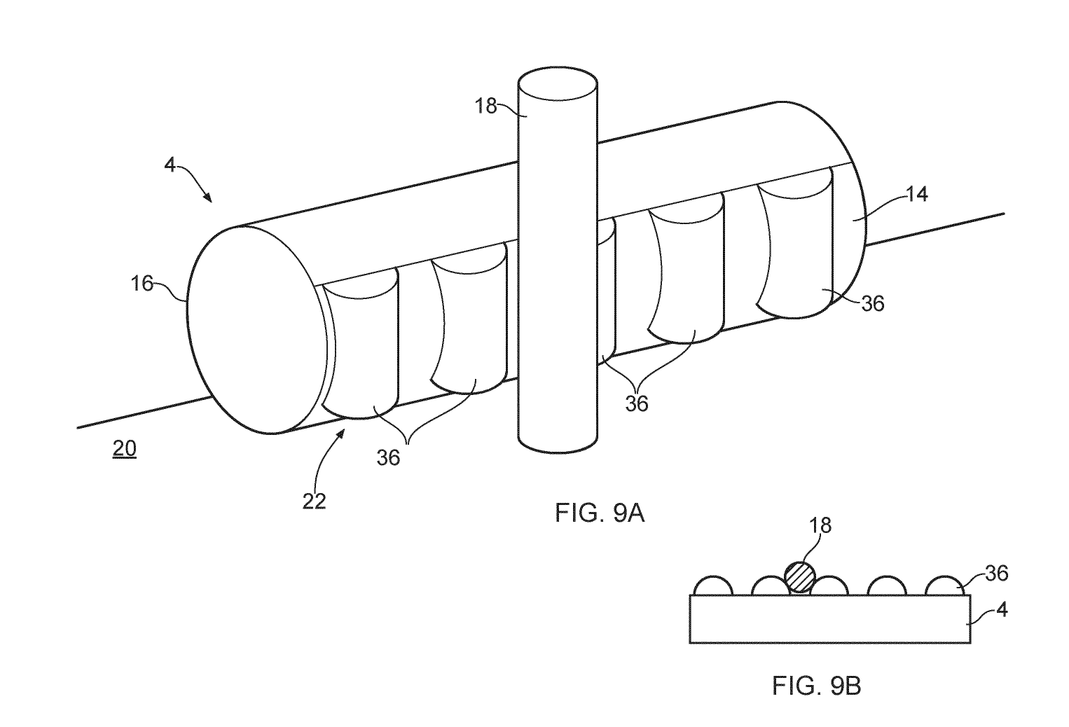

FIG. 9 shows a cutting element 4 which includes a plurality of ridges in the form of half cylinders 36. The ridges 36 in this embodiment have semi-circular cross sections. As with the ridges of other embodiments discussed above, the semi-cylindrical ridges 36 serve, in use, to guide a hair 18 towards the cutting face 14 in the region between adjacent ridges, and restrict lateral movement of the hair while in contact with the cutting face.

The movement restrictors and/or the ridges of any of the hair support structures described herein may be formed from a material suitable for contacting the skin of a user in a shaving environment. For example, a material may be chosen which is easy to clean, which moves over skin easily with a relatively low amount of friction, which is non-allergenic, which non-corrosive and/or which is suitable for use in a wet environment. In some embodiments the hair support structure 22 may be formed at least in part from metal, such as stainless steel, from a plastics material or from a polymer.

As noted above, it may be advantageous for the movement restrictors and the ridges of the hair support structure 22 to be formed from a material which is enables ends of the movement restrictors to be temporarily laterally displaced when moved into contact with an object. However it may also be advantageous for the hair support structure 22 to be formed from a material strong enough such that in use, the movement restrictors apply pressure onto a user's skin, so as to manipulate the skin from which the hairs to be cut are growing. In this way, the hair support structure 22 may push hairs out the skin to cause an improved closeness in the shaving or hair cutting activity.

The arrangements shown in FIGS. 4 to 10 are merely exemplary and the hair support structure 22 may have more or fewer movement restrictors than is shown. The number of movement restrictors (i.e. protrusions and/or ridges) forming the hair support structure 22 may depend at least in part on the length of the cutting element 4, the type of hair to be cut by the cutting element, the size of the movement restrictors, and the desired separation between adjacent movement restrictors. For example, hairs growing on legs of a human might have a diameter of between around 50 micrometres and 80 micrometres, whereas facial hairs of a human, such as beard hair might have a diameter of between around 150 micrometres and 400 micrometres. In some examples, therefore, a separation between adjacent movement restrictors and/or ridges may be between approximately 20 micrometres and 500 micrometres. More specifically, the separation may be between approximately 50 micrometres and 400 micrometres. The term “separation” in this context may be considered to be the distance between portions of adjacent movement restrictors where a hair 18 is to be positioned while being cut (i.e. while the hair is touching the cutting face 14).

The hair support structure 22 may be coupled to the cutting element 4, for example using adhesive. In some embodiments, the hair support structure 22 may be attached to the cutting face 14, while in other embodiments, the structure 22 may be attached to another part of the cutting element 4, remote from the cutting face. By attaching the hair support structure 22 to the cutting element 4, rather than forming the structure 22 as part of the optical waveguide, the cutting face remains intact, such that light propagating through the optical waveguide is able to couple out from the cutting face in a consistent and predictable manner. Changing the structure of the cutting face 14 itself may affect the coupling of light from the coupling element 4 into a hair 18.

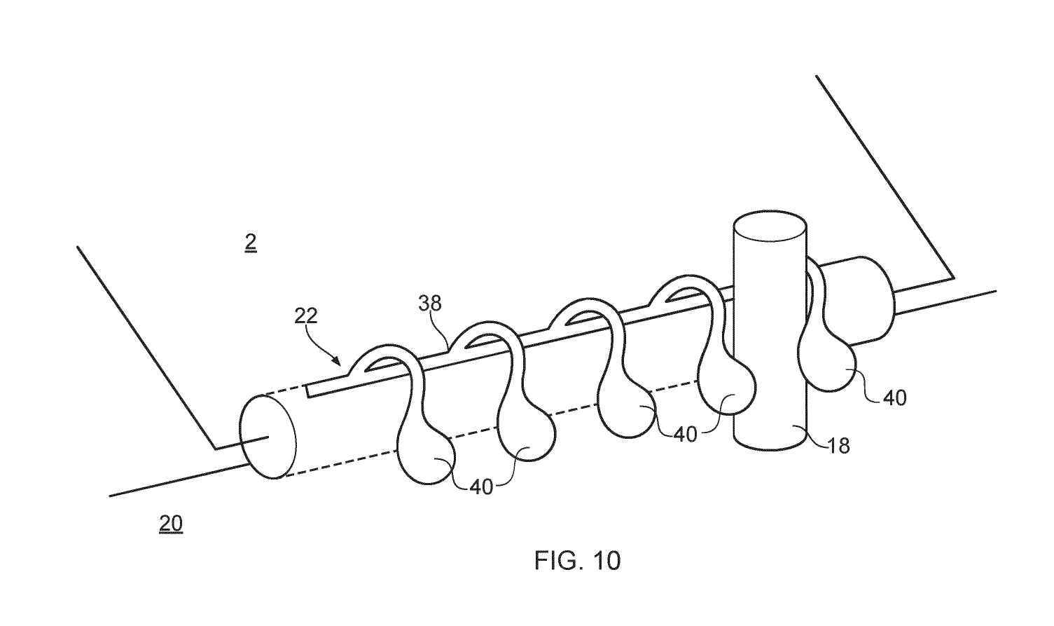

In some embodiments, the hair support structure 22 may be removably attachable to the cutting element 4 or to the hair cutting device 2. FIG. 10 shows an example hair support structure 22 having an attachment assembly 38. Portions of the cutting element 4 in FIG. 10 are shown in dashed lines for improved clarity. In this example, the hair support structure 22 includes bulbous protrusions 40 similar to the protrusions 28 shown in FIG. 6. However, the attachment assembly 38 may be incorporated into any hair support structure, such as those described herein. The attachment assembly 38 shown in FIG. 10 functions as a clip to enable the hair support structure 22 to be clipped onto the cutting element 4 or onto a portion of the hair cutting device 2. In other embodiments, the attachment assembly 38 may enable the hair support structure 22 to be removably attached to the cutting element 4 or to the hair cutting device 2 in another way. By forming the hair support structure 22 such that it can be removably attached to the cutting element 4, or demountable, a user may be able to remove the hair support structure quickly and easily, for example to replace it with a different support structure, suitable for cutting a different type of hair. In some embodiments the hair support structure 22 may be disposable, or removable for cleaning, for example.

In some embodiments, the cutting face 14 comprises a surface of the cutting element 4, such as, for example, an outer surface of a cladding of an optical fibre. In some embodiments, a portion of the cladding of the optical fibre is removed, such that the cutting face 14 comprises a surface of the core of the optical fibre 4. In either of these scenarios, the cutting face is uninterrupted, with no intended recesses, indentations or protrusions, other than the hair support structure 22 coupled thereto.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage.

Any reference signs in the claims should not be construed as limiting the scope.

1. A cutting element for use in a hair cutting device, the cutting element comprising:
an optical waveguide having a sidewall, wherein a portion of the sidewall forms a cutting face for contacting hair; and
a hair support structure configured to restrict lateral movement of a hair in contact with the cutting face, relative to the optical waveguide,
wherein the hair support structure comprises a plurality of movement restrictors extending from the cutting face, and
wherein each movement restrictor comprises a ridge protruding from the cutting face of the optical waveguide, the ridges being arranged substantially parallel to one another.

2. A cutting element according to claim 1, wherein each movement restrictor is shaped substantially in a shape selected from a group comprising: conical, cubic, cylindrical, teardrop-shaped and bulbous.

3. A cutting element according to claim 1, wherein each ridge comprises a structure shaped substantially in a shape selected from group comprising: a cuboid, a triangular prism and a half cylinder.

4. A cutting element according to claim 1, wherein a separation between adjacent movement restrictors is between around 20 micrometres and 500 micrometres.

5. A cutting element according to claim 1, wherein, in use, the hair support structure is arranged to engage skin of the user from which extends the hair to be cut.

6. A cutting element according to claim 1, wherein the hair support structure is coupled to the optical waveguide using adhesive.

7. A cutting element according to claim 1, wherein the hair support structure is configured to be connected to the optical waveguide by an attachment assembly.

8. A cutting element according to claim 1; wherein the optical waveguide comprises an optical fibre.

9. A hair support structure for use with a cutting element, the cutting element comprising an optical waveguide having a sidewall, wherein a portion of the sidewall forms a cutting face for contacting hair;
wherein the hair support structure comprises a plurality of movement restrictors, and
wherein each movement restrictor comprises a ridge protruding from the cutting face of the optical waveguide, the ridges being arranged substantially parallel to one another such that the hair support structure is configured to restrict lateral movement of a hair in contact with the cutting face, relative to the optical waveguide.

10. A hair support structure according to claim 9, further comprising:
an attachment assembly for removably attaching the hair support structure to a cutting element or to a hair cutting device including a cutting element.

11. A hair support structure according to claim 10, wherein the attachment assembly comprises a clip for clipping the hair support structure onto a cutting element or a hair cutting device.

12. A hair cutting device for cutting hair on a body of a subject, the hair cutting device comprising:
a light source for generating laser light at one or more specific wavelengths corresponding to wavelengths absorbed by one or more chromophores in hair; and
a cutting element coupled to the light source to receive laser light, the cutting element comprising a cutting element according to claim 1.

13. A hair cutting device according to claim 12, wherein the hair support structure is removably attachable to the cutting element.
